(19) United States
(12) Patent Application Publication
(10) Pub. No.: US 2026/0224455 A1
(43) Pub. Date: Aug. 6, 2026

(54) HAIR COLORANT CONTAINING BIOACTIVE GLASS

(71) Applicant: SCHOTT AG, Mainz (DE)

(72) Inventors: Thomas MAIER, Landshut (DE); Antonio TRIZZINO, Landshut (DE); Matthias RINDT, Landshut (DE); Eva MUELLER, Bad Kötzting (DE); Selina REIGL, Abensberg (DE)

(21) Appl. No.: 19/132,912

(22) PCT Filed: Nov. 28, 2023
(86) PCT No.: PCT/EP2023/083252

§ 371 Date: May 27, 2025

(30) Foreign Application Priority Data
Nov. 29, 2022 (DE) 10 2022 131 545.2

(51) Int. Cl. A61K 8/25; A61K 8/19; A61K 8/24; A61Q 5/06; A61Q 9/02; A61Q 9/04

(52) U.S. Cl. CPC A61K 8/25; A61K 8/19; A61K 8/24; A61Q 5/065; A61Q 9/02; A61Q 9/04; A61K 2800/30; A61K 2800/43; A61K 2800/651

(57) ABSTRACT

Colorants, in particular hair colorants, frequently contain ammonia, which leads to an unpleasant odor during use and can irritate skin, hair and the lungs. In the case of known alternatives to ammonia, the coloring performance or the durability of the color is frequently weaker. This problem is solved by proposing a colorant which comprises an alkalizing agent to open the cuticle layer of hair and/or to swell hair and/or skin, wherein the alkalizing agent comprises or consists of particles of bioactive glass as alkalizing agent.

The invention relates to a colorant, in particular hair colorant, and to the use of an alternative alkalizing agent in a cosmetic formulation, in particular a colorant for coloring keratin fibers.

Keratin fibers are the main constituent of mammalian hair such as wool, fur, feathers, human hair, and skin. Where reference is made hereinafter to hair colorants, this is for reasons of linguistic simplification, without being limited to hair. In addition, the term “hair colorant” is used as a generic term for “hair colorant in the literal sense, i.e. for coloring hair” and for “skin colorant for coloring skin”. The term “coloring” is broadly understood in the context of this invention in the sense of “changing the color” and includes adding a color to the keratin fiber and reducing colors present in the keratin fiber.

Colorants for coloring keratin fibers, in particular hair and/or skin, can be divided into different groups, particularly with regard to their mechanism of coloring and durability when washed, for example with surfactant-containing detergents. The different groups are described hereinbelow using hair colorants as an example. The same applies to skin colorants with which keratins present in skin are dyed.

Where general reference is made hereinafter to a “dye”, this term encompasses dyes in the narrower sense, i.e. soluble dyes, and insoluble pigments. Persons skilled in the art know which dye species are to be employed in the various colorants.

Hair colorants, their different action principles, and suitable dyes are generally known, for example S. A. Da Franca, M. F. Dario, V. B. Esteves, A. R. Baby, M. V. R. Velasco, “Types of Hair Dye and Their Mechanisms of Action”, Cosmetics 2015, 2(2), 110-126.

In the case of non-oxidative hair colorants, hair coloring is effected by dyes, for example what are known as direct dyes, that, on account of their positive charge, become attached only superficially to the negatively charged outer sheath of the hair (temporary coloring) or, on account of their small molecular size, penetrate into the cuticle layer of the hair and adhere to the hair keratin (semi-permanent coloring). In the case of non-oxidative hair coloring, the natural pigments of the hair are not destroyed or chemically altered, but are overpowered by the applied dyes.

Temporary coloring is based primarily on an electrostatic attraction between the negatively charged outer sheath of the hair and usually cationic dyes that are supplied via a colorant preparation. However, this interaction is not strong and the attachment can be curtailed through subsequent hair washes. This mechanism of action differs significantly from semi-permanent or permanent hair coloring.

In the case of semi-permanent coloring, dye molecules or pigments not only adhere to the surface of the hair but on account of their small size also diffuse into the outer cuticle layer of the hair, also termed the “cuticula”. There, they are however bound—for example through charge—only weakly or not at all, which is why the durability of the colors is limited to a few hair washes—up to a maximum of about 12 hair washes, depending on the hair structure and dye. Semi-permanent colorants also include natural colorants based on constituents of plant origin, such as henna, chamomile, bark extracts, etc.

Temporary colorants and semi-permanent colorants are colorants for physically changing the color, since the dyes are physically bound to and/or in the hair.

In the case of permanent colorants, the natural pigment of the hair is also chemically altered, the change of color being permanent, i.e. it cannot be washed out through normal hair washes. Permanent coloring of hair usually employs an oxidizing process, which is why the colorant preparations used are also termed oxidation colorants or oxidative colorants. In the context of the invention, oxidation hair colorants also include lightening colorants and bleaching agents, which lighten or oxidatively destroy the natural pigments in order to produce a blond shade, and demi-permanent hair colorants, which combine the functionality of permanent and semi-permanent coloring and are referred to also as “intensive tinting” or “coloring”. Other concepts for permanent coloring proposed in the literature are primarily ones based for example on reactive dyes that bind to functional groups in hair, or else on the formation of azo dyes from precursors.

A permanent colorant can be a one-component colorant or a multicomponent colorant, in particular a two-component colorant; what they all have in common is that an alkalizing agent for opening the hair structure and an oxidant for altering the natural hair pigments and/or for forming or binding the desired color are required. Oxidation colorants are employed because of their intense colors and long durability of the coloring result, the coloring mechanism being sufficiently well known (see, for example, O. J. X. Morel, R. M. Christie, “Current Trends in the Chemistry of Permanent Hair Dyeing”, Chem. Rev. 2011, 111, 2537-2561.; A. Towns, “A review of developments in industrial hair colorant actives for oxidative dyes”, Coloration Technology 2021, 137, 301-335.

In the context of use of a one-component colorant, it is for example possible to apply to the hair at least one dye, for example a dye containing synthetic melanin or another easily oxidizable dye, that reacts with e.g. atmospheric oxygen either on its own or with the aid of a catalyst (for example manganese sulfate) to form nature-like dyes, which can be advantageous, particularly for graying hair. Such colorants are also termed “auto-oxidative colorants” and do not require such high pH values when coloring. However, the higher the pH, the more rapidly and the more deeply the dyes penetrate into the hair and do not wash out so quickly. This mechanism of action has its limits in people with dark hair (red, brown, black). In their case, oxidation dye precursors are necessary, as described below.

Oxidation colorants/permanent colorants are usually multicomponent products, commonly two-component products, in which active ingredients, auxiliaries, and additives are distributed among two or more components that are mixed together to form the ready-to-use colorant. In a two-component product, “component 1” (also termed “coloring component”) includes at least one coloring agent and “component 2” (also termed “oxidation component”) includes an oxidant for forming the color. The components, in particular the coloring component and the oxidation component, must be mixed together before use.

Oxidation colorants cause a chemical change of color and contain various active ingredients, auxiliaries, and additives. Examples of auxiliaries and additives include viscosity adjusters, vehicle compounds, solubilizers, fragrances, etc.

Coloring agents are an active ingredient normally present in the coloring component of the colorant: an exception here may be so-called bleaching agents (see below). Coloring agents, also referred to as “coloring components”, include in the case of oxidation colorants in particular color precursors, which are also referred to as color bases and have the characteristic feature of being easily oxidizable, optionally also nuancing agents (i.e. dyes or pigments that alter the shade a little), optionally also direct dyes, as is generally well known to persons skilled in the art.

The often only weakly colored or colorless color precursors are able to penetrate into the interior of the hair on account of their small size. Changing the color chemically is also termed oxidative coloring, since “developing” the color requires an oxidant, for example hydrogen peroxide, atmospheric oxygen, special enzymes, amino compounds, etc. Under the influence of the oxidant, the color precursors give rise themselves, or through coupling with one or more coupler components, to the actual dyes or color pigments, which, on account of their size and poor water solubility, can no longer be washed out of the hair.

Oxidants, in particular hydrogen peroxide, can have a dual function in permanent colorants, particularly when used in relatively high concentration (for example, hydrogen peroxide content>4% by weight). A part of the hydrogen peroxide causes lightening of the natural or synthetic hair pigments, while the other part is needed for the coloring process, i.e. the formation of synthetic dyes in the hair. At a lower hydrogen peroxide content (for example 1 to 4%), the lightening effect is lower and essentially only the synthetic dyes are formed in the hair. In a two-component colorant, the oxidizing active ingredient is usually present in the second component of the colorant.

To achieve optimal coloring results, oxidation colorants are adjusted to alkaline pH in the 8 to 11 range, for which an alkalizing agent is present in the colorant as active ingredient, usually in the coloring component in the case of a two-component colorant. The alkalizing agent effects opening of the cuticle layer and swelling of the hairs, as a result of which the coloring components, for example color precursors, nuancing agents, and direct dyes, are able to penetrate into the interior of the hair. There, they are oxidized by reaction with an oxidant to form complex dyes or larger oligomers, which then become insoluble or are prevented from diffusing on account of their size. Some dyes produced are able to combine chemically with the hair keratin. The oxidation of the color precursors with the oxidant results in formation of the dye or synthetic pigment. An increased pH is often also necessary for the desired oxidation reaction.

The pH in known colorants can be adjusted using ammonia as alkalizing agent. A drawback of known permanent colorants that employ an ammonia-based alkalizing agent (for example ammonium hydroxide) is the unpleasant odor caused by ammonia during use. Ammonia gas released during use can irritate the skin and eyes, and the lungs if inhaled. In addition, the customary alkalizing agent has an adverse effect on the structure of the hair, since swelling of the hairs also allows other molecules to penetrate into the interior of the hairs and accordingly has a potentially allergenic effect. The same applies to ammonia-based skin colorants.

There are already ammonia-free oxidation colorants that use for example amines as alkalizing agents, for example monoethanolamine (e.g. 2-ethanolamine) on its own or in combination with other alkalizing agents such as amino acids, oligopeptides or acylamino acid derivatives, etc., as disclosed for example in EP 2 178 492 B1, US 2014/0082856 A1, US 2012/0180231 A1, DE 19527121 A1, but their coloring power can be weaker compared to NH3-based oxidation colorants. In addition, the durability of the color in hair has been found to be lower, which means that the hair must be dyed more often.

The object of the invention is to provide a colorant, in particular a hair colorant, that does not have the abovementioned disadvantages. The colorant should have no unpleasant odor and minimal irritating properties and advantageously have high coloring power, color intensity, and durability in respect of being washed out and exposed to light. An additional object of the invention is to provide a substitute for known alkalizing agents in a formulation for cosmetic products, in particular for coloring keratin fibers.

### DISCLOSURE OF THE INVENTION

The above object is according to a first aspect of the invention achieved by a colorant, in particular hair colorant, that comprises an alkalizing agent for effecting opening of the cuticle layer of hairs and/or swelling of hairs and/or skin, the alkalizing agent comprising particles of bioactive glass as alkalizing agent or consisting thereof.

In the case of oxidative (hair) colorants, the particles of bioactive glass as alkalizing agent can also have the function of catalyzing or initiating coupling reactions of/with color precursors.

It is preferable that a colorant of the invention is suitable for coloring human hair and/or skin and is therefore a hair colorant, it being possible for the term “hair colorant” according to the definition given in the introduction to also mean a “skin colorant”. The colorant can however also be used for coloring other keratin fibers, for example fur, wool, and feathers, and also for coloring other substrates.

An advantageous colorant colors as hair colorant in the literal sense the hair. An advantageous colorant colors as skin colorant the skin or, depending on where it is applied, additionally colors the hair present there. For example, a skin colorant of the invention can be used to selectively color the skin in the region of the eyebrows and thereby alter the visual impression, for example to simulate denser hair growth.

As alkalizing agent, the colorant according to the invention includes particles of bioactive glass or consists thereof. Particles of bioactive glass means that the particles include bioactive glass or consist thereof. In the context of the invention, it has been found that particles of bioactive glass can surprisingly be used as alkalizing agent in a colorant, since in an aqueous medium they are reactive, release ions, and accordingly raise the pH. As alkalizing agent in a hair colorant, they effect opening of the cuticle layer of the hairs and cause them to swell. As alkalizing agent in a skin colorant, it causes the skin to swell. Thus, in the context of the invention, particles of bioactive glass assume the function of ammonia and/or known ammonia alternatives. In other words, in the context of the invention, particles of bioactive glass are a substitute for ammonia and/or ammonia alternatives as alkalizing agent, as is described in more detail below. This firstly reduces the problem of the unpleasant odor and irritation of the skin, eyes, and lungs during use. Secondly, bioactive glass can be used to selectively adjust the prevailing pH on the hair or skin, for example by varying the use concentration, as a result of which coloring is gentler for the skin and hair.

Ammonia and ammonia alternatives are known alkalizing agents in colorants, in particular hair colorants. “Ammonia alternatives” are understood to mean agents that in a colorant have an effect corresponding to the ammonia, i.e. are equivalent in effect. Ammonia alternatives can preferably be selected from the group including amines, for example monoethanolamine (e.g. 2-ethanolamine), amino acids, oligopeptides, and acylamino acid derivatives.

DE 102007033091 A1 mentions for example the following alkalizing agents: ammonia, organic amines, such as monoethanolamine, monoisopropanolamine, 2-amino-2-methylpropanol, 2-amino-2-methylpropane-1,3-diol, 2-amino-2-ethylpropane-1,3-diol, 2-amino-2-methylbutanol, triethanolamine, and ammonium, alkali metal, and alkaline earth metal hydroxides, carbonates, hydrogen carbonates, hydroxycarbonates, and silicates, in particular metasilicates, and alkali metal phosphates.

Whereas known alkalizing agents determine the pH of the colorant as a whole, a pH of 8 to 11 usually being established, an advantageous colorant comprising particles of bioactive glass can have a neutral pH, this being understood in the context of the invention to mean a pH within a range of 6 to <8, preferably 6.5 to 7.5. This is possible because bioactive glass is reactive and special particles of bioactive glass in contact with the hair and/or skin locally make the pH more basic and in doing so effect opening of the cuticle layer of the hairs and cause hair and/or skin to swell and/or enable reactions resulting in color formation, while the pH of the colorant is neutral overall. In such variants, the particles of bioactive glass act as a local alkalizing agent and the particles of bioactive glass are thus advantageously a local alkalizing agent. The term “alkalizing agent” in relation to particles of bioactive glass thus does not imply that the colorant must have a basic pH overall when applied, unlike with known alkalizing agents. This effect is assumed to be caused or promoted by the fact that particles of bioactive glass have a high affinity for keratin fibers, with the result that the pH is increased in particular locally on the hair or locally on the skin, while the pH of the colorant is overall neutral or only slightly alkaline. This may result in less skin irritation when coloring with the advantageous colorant. The coloring performance of an advantageous colorant having a neutral pH and the color durability over multiple washes with standard commercial shampoo are surprisingly comparable to the results of conventional hair colorants based on ammonia or ammonia alternatives, in which the coloring process takes place at alkaline pH>10.

In an advantageous alternative it is of course possible for the colorant also to have an alkaline pH overall, in particular in the 8 to 11 range, preferably 9 to 11 or 8 to 10.

In an advantageous embodiment, particles of bioactive glass as alkalizing agent may be present in the colorant in addition to another alkalizing agent, for example ammonia and/or ammonia alternatives, in order to reduce the proportions thereof. Ammonia and/or ammonia alternatives are accordingly only partly replaced.

In an advantageous embodiment, the total amount of ammonia and ammonia alternatives in the colorant is less than 5% by weight, preferably less than 3% by weight, preferably less than 2% by weight, preferably less than 1% by weight.

In a particularly advantageous embodiment, particles of bioactive glass are used as sole alkalizing agent. In other words, the alkalizing agent in this variant of the colorant consists of particles of bioactive glass, i.e. the alkalizing agent is bioactive glass. There is therefore a complete replacement of ammonia and/or ammonia alternatives.

In a particularly advantageous variant, the colorant is free of ammonia and/or free of ammonia alternatives. “Free of” in this context means that said constituents are not actively added to the colorant, but may be present in at most unavoidable traces or as contaminants, which may be due for example to impurities in the ingredients used. An impurity is understood to mean a content of not more than 100 ppm, preferably a content of not more than 50 ppm for ammonia or a content of not more than 100 ppm, preferably a content of not more than 50 ppm, for each ammonia alternative. Particular preference is of course given to complete freedom from said undesirable substances in the colorant.

Experiments with advantageous hair colorants comprising particles of bioactive glass as sole alkalizing agent have shown that the coloring performance of the colorant and color durability over multiple washes with standard commercial shampoo are at least as good as that with known hair colorants based on a conventional alkalizing agent and are in some cases even better, for example when there is a higher content of particles of bioactive glass.

The reactivity of particles of bioactive glass and thus the prevailing pH in the colorant when applied to hair and/or skin can be selectively adjusted by a number of measures, which are described in more detail hereinbelow.

In an advantageous variant, the colorant has an alkaline pH in the 8 to 11 range, preferably 9 to 11 or 8 to 10. In an alternative advantageous variant, the colorant has a neutral pH, preferably 6 to <8, preferably 6.5 to 7.5.

In an advantageous embodiment of the colorant, the colorant comprises at least one coloring component and/or at least one oxidant.

In an advantageous development, the colorant includes at least one coloring component. The term “coloring component” encompasses in principle all known coloring agents used in colorants (dyes, pigments, color precursors, etc.) that are suitable for temporary, semi-permanent, demi-permanent or permanent coloring of keratin fibers, in particular hair and/or skin. Suitable in the context of the invention are those coloring components that can be used alongside particles of bioactive glass as alkalizing agent for effecting opening of the cuticle layer of hair and swelling of hair and/or skin. These can be, for example, ready-to-use dyes (also termed “direct dyes”) or color precursors (also termed “undeveloped dyes” or “dye bases”), the colored synthetic dyes or pigments being formed from the color precursors only by means of an oxidant. Coloring components also include nuancing agents (also termed color couplers), which are partially developed or unoxidized color formers that give a color a particular color direction, and also plant dyes.

In an advantageous embodiment of the colorant, it is a colorant selected from the group comprising temporary colorants, semi-permanent colorants, demi-permanent colorants, and permanent colorants, wherein these may preferably be colorants for hair and/or skin. Persons skilled in the art know which types of coloring components are to be used in each of the different types of colorant.

Examples of coloring components and the different mechanisms of action of coloring are described in numerous publications, for example in S. A. Da Franca, M. F. Dario, V. B. Esteves, A. R. Baby, M. V. R. Velasco, “Types of Hair Dye and Their Mechanisms of Action”, Cosmetics 2015, 2(2), 110-126, the disclosure content of which is included in its entirety.

Examples of coloring components, in particular for permanent hair colorants, are described inter alia in O. J. X. Morel, R. M. Christie, “Current Trends in the Chemistry of Permanent Hair Dyeing”, Chem. Rev. 2011, 111, 2537-2561; A. Towns, “A review of developments in industrial hair colorant actives for oxidative dyes”, Coloration Technology 2021, 137, 301-335, the disclosure content of which is included in its entirety.

Coloring components are often based on substituted aromatic compounds such as phenol, aniline, and toluene. The predominant color precursors on the market are for example 2,5-diaminotoluene in the form of the sulfate, p-phenylenediamine, p-aminophenol, and 1-hydroxyethyl-4,5-diaminopyrazole in the form of the sulfate.

In an advantageous embodiment of the colorant, it is a non-oxidative colorant in which particles of bioactive glass act as an alkalizing agent that causes the cuticle layer of the hairs to open and the hair and/or skin to swell, with the result that at least one coloring component, for example a nonionic dye, is able to penetrate more easily and more deeply into hair and/or skin. This can improve the color intensity and durability of the color toward washing in the case of temporary and/or semi-permanent colorants.

In an advantageous embodiment, the colorant is a permanent colorant that brings about a permanent chemical change of color. Permanent colorants include the group of demi-permanent colorants, since the coloring mechanism is based on the same principle. However, the coloring effect and durability are somewhat weaker in the case of demi-permanent colorants. Preferably, it is an oxidation colorant that requires an oxidant for generating the synthetic dyes or pigments and/or for altering the natural color pigments in hair and/or skin.

In an advantageous embodiment of the colorant, the colorant comprises at least one oxidant and is thus an oxidation colorant. In an advantageous variant, the oxidation colorant is a bleaching agent that includes hydrogen peroxide and/or another oxidant for lightening the natural hair pigments. The particles of bioactive glass cause the cuticle layer of the hair to open, with the result that the hair swells and the natural hair pigments or synthetic pigments of prior colorings are lightened or broken down, for example by hydrogen peroxide. Advantageous oxidants and proportions are described further below. Bleaching agents may also comprise coloring components to avoid undesired color impressions. The principal focus of these agents is however the lightening or destruction of the natural hair pigments, it being possible to control the intensity of lightening through the concentration of the oxidant, in this case in particular hydrogen peroxide. The same applies to lightening colorants.

In an advantageous embodiment of the colorant, the colorant comprises at least one coloring component and at least one oxidant. The oxidation colorant here comprises a coloring component, through which color depth (lighter, darker) and/or color direction of hair and/or skin can be altered selectively and permanently.

In a first advantageous variant of the oxidation colorant comprising a coloring component, it is a one-component colorant in which particles of bioactive glass as alkalizing agent cause the cuticle layer of the hairs to open and the hair and/or skin to swell. This allows a coloring component to become embedded in the keratin fibers and oxidized to form nature-like dyes, for example by atmospheric oxygen or a catalyst.

In another advantageous variant of the oxidation colorant comprising a coloring component, it is a multicomponent colorant wherein active ingredients, auxiliaries, and additives are distributed among two or more components that are mixed together upon application of the colorant. In this case, one component contains at least one coloring component and preferably particles of bioactive glass as alkalizing agent for effecting opening of the cuticle layer of hairs and swelling (as described above), and another component contains an oxidant. It is however also possible for the alkalizing agent to be present in a different component. Multicomponent colorants are known to persons skilled in the art, who know how to formulate the individual components and assemble them for use.

The component comprising the oxidant can advantageously comprise 0.5% to 12% by weight of hydrogen peroxide. The hydrogen peroxide content should be chosen according to the type of oxidative colorant, as is known to persons skilled in the art. For example, bleaching agents may contain up to 12% by weight of hydrogen peroxide, whereas other permanent colorants may contain up to 9% by weight or up to 6% by weight depending on the degree of desired lightening. For weaker colorants, for example demi-permanent colorants, an upper limit may also be 4% by weight of hydrogen peroxide. Alternatively or in addition, other oxidants may also be present in the component, for example sodium iodate or sodium periodate, products of hydrogen peroxide addition to, for example, urea, melanin, borates, etc. Hydrogen peroxide variants and hydrogen peroxide substitutes are well known in the specialist field.

In an advantageous variant, the colorant may be a two-component colorant wherein “component 1”, also termed “coloring component”, comprises the at least one coloring component, and “component 2”, also termed “oxidation component”, includes an oxidant for forming the color. “Component 1” and “component 2” must be mixed together before use and together give rise to the ready-to-use colorant. The particles of bioactive glass as alkalizing agent may be present together with at least one coloring component in “component 1” of the colorant.

In an advantageous embodiment, the multicomponent colorant, for example two-component colorant, includes as coloring component color precursors, i.e. dye precursors, for example easily oxidizable compounds that on account of their small molecular size are able to readily penetrate deeply through the opened cuticle layer into the swollen hair or into the skin. The presence of an oxidant, in particular hydrogen peroxide and/or known alternatives, for example sodium iodate, sodium periodate, etc. (see above), triggers the oxidative process, wherein the color precursors are developed by the active oxygen to form dye(s). In an advantageous variant, the coloring component comprises in addition to color precursors at least one further dye, for example a direct dye.

The colorant according to the invention comprises particles of bioactive glass. The median diameter of the particles, i.e. the d₅₀ value, is advantageously≤20 μm, advantageously≤15 μm, preferably ≤10 μm, preferably ≤5 μm. The smaller the particle size, the greater the reactive surface area of the particles and the more strongly or more rapidly the pH can be shifted to alkaline.

A lower limit for the d₅₀ value of advantageously 0.1 μm, preferably 0.25 μm, which can be combined with any of the above upper limits, should not be gone below.

The d₉₉ value of the particle size is in an advantageous variant≤60 μm, advantageously≤50 μm, advantageously≤40 μm, advantageously≤30 μm, advantageously≤25 μm, advantageously≤20 μm, preferably ≤15 μm. A lower limit for the d₉₉ value, which can advantageously be combined with any of the above upper limits, can be 1 μm. The d₉₉ value is the value at which 99% of the measured particles have a diameter equal to or smaller than the stated value.

The particle size (grain size) is determined by laser diffraction according to ISO 13320:2020 and is based on the phenomenon that the angular distribution of the intensity of light scattered by a particle is dependent on the particle size, which is subsequently described as the diameter of an assumed sphere-like shape. The theoretical basis employed is Fraunhofer diffraction or Mie scattering, depending on the particle size.

For the purposes of the present description, the term “diameter” refers to the maximum extent of the particle. In the case of spherical particles, the diameter simply corresponds to the diameter of the sphere. In the case of ellipsoidal or plate-like particles, the diameter is measured at the point of its maximum extent, for example along its main axis in the case of an ellipsoid. It is not possible to make any statement on the shape of the particles, since this information is not provided by laser diffraction.

The particles of bioactive glass, i.e. a powder of bioactive glass, can be produced in a known manner, for example by a dry grinding or wet grinding process or a combination of the two. The process for producing a glass powder can be carried out both dry and with aqueous and non-aqueous grinding media. The milling tool can include inter alia a ball mill, agitator ball mill, jet mill, pin mill or a combination of two or more thereof. This makes it possible to produce a material having an advantageous particle size as described above.

With regard to particle size, it is advantageous if the particles do not undergo sedimentation in the colorant. It may be advantageous to choose the particle size according to the viscosity of the colorant or to bring the viscosity in line with a desired particle size through appropriate additives, for example thickeners (e.g. xanthan gum).

In an advantageous embodiment, the proportion of particles of bioactive glass in the colorant is 0.1% to 20% by weight, advantageously 0.5% to 15% by weight, advantageously 0.7% to 10% by weight. The proportion in the case of a one-component colorant refers to the ready-to-use colorant. In the case of a multicomponent colorant, in particular a two-component colorant, the proportion refers to the coloring component of the colorant, for example to “component 1”.

The proportion of particles of bioactive glass in the colorant can advantageously be at least 0.1% by weight in order to obtain the desired alkalizing effect in the colorant. An advantageous lower limit can be at least 0.3% by weight, at least 0.5% by weight, at least 0.7% by weight, at least 0.9% by weight or at least 1% by weight. Some variants may also comprise at least 1.5% by weight or at least 2% by weight of particles of bioactive glass. An upper limit of not more than 20% by weight should advantageously not be exceeded, since otherwise the alkalizing effect is too strong, which can result in skin irritation as well as hair and skin damage. In addition, the bioactive glass particles can influence the rheological properties, in particular the viscosity, of the colorant, with the viscosity rising or falling according to the composition of the original color and thus making the colorant easier or more difficult to apply using for example self-operated manual-pressure applicators. An advantageous upper limit can be not more than 15% by weight, not more than 12% by weight, not more than 10% by weight, not more than 8% by weight, not more than 6% by weight or not more than 5% by weight.

As described above, particles of bioactive glass may be employed as sole alkalizing agent or in a mixture with other alkalizing agents, i.e. ammonia and/or ammonia alternatives.

The proportion of particles of bioactive glass necessary in a colorant depends inter alia on the type of colorant, the reactivity of the bioactive glass, the particle size, and the desired pH during coloring.

In an advantageous development, the colorant contains particles of ceramized bioactive glass, i.e. particles of a bioactive glass ceramic. Glass ceramic is understood to mean a material that has an amorphous phase and a crystalline phase. Through selective formation of particular crystal phases and the proportions of the crystalline phases and the amorphous phase, it is possible to control the reactivity of the bioactive glass ceramic, i.e. the rate of ion exchange with a liquid medium and thus the establishment of the pH. The higher the reactivity, the fewer particles/less powder required to establish the desired pH. Particles of a bioactive glass ceramic are described in more detail further below.

In an advantageous variant, particles of a bioactive glass ceramic may be present in the colorant in addition to particles of bioactive glass. Alternatively, it is also possible for all particles to be ceramized, i.e. present as bioactive glass ceramic.

The fact that the particles are of bioactive glass or of a bioactive glass ceramic does not necessarily mean that the particles consist 100% of glass or glass ceramic, although this may be the case in some embodiments of the invention. In an advantageous embodiment of the invention, the surface of the particles may have undergone chemical modification, including in particular a functionalization. For example, the particles may have properties that are more hydrophilic or more hydrophobic as a result of reaction with appropriate silanes. This makes it possible to adjust the reactivity in a water- or oil-based formulation.

The following embodiments concern bioactive glass or particles of bioactive glass. The description of the particles of bioactive glass of the invention and of the advantageous developments thereof relates both to colorants of the invention comprising particles of bioactive glass and to the use according to the invention of particles of bioactive glass as alkalizing agent in a cosmetic formulation.

Bioactive glass refers to a glass containing SiO₂ as glass former and at least one oxide selected from alkali metal oxide and alkaline earth metal oxide as network former, wherein the bioactive glass displays a specific biological reaction upon contact with an organism. When it comes into contact with a fluid, such as a bodily fluid, the bioactive glass exchanges ions with the fluid. In this process, monovalent alkali metal oxide ions and/or divalent alkaline earth metal oxide ions, for example and depending on the composition Na₂O in particular, are released from the glass and a SiO₂-rich layer, in particular a silica gel layer, forms on the glass surface within a few minutes. When the bioactive glass also advantageously comprises P₂O₅ and CaO, a hydroxyl-carbonate-apatite layer very similar to the mineral phase of bone can form on the glass surface. Such bioactive glasses are accordingly able to form a permanent physicochemical bond to bone and tissue and are not encapsulated or rejected by the body.

Bioactive glasses have long been known and a summarizing description is given for example by L. L. Hench, J. K. West, “Biological Applications of Bioactive Glasses”, Life Chemistry Reports, 1996, 13, 177-241. In contrast to conventional non-bioactive glasses, bioactive glasses have the characteristic feature of being soluble in an aqueous medium.

Bioactive glasses are proposed for example for use in toothpastes for remineralization of teeth and preparations for sealing open dentine tubules, in materials for bone regeneration, in preparations for inflammation control, for example in poorly healing wounds or acne, as a mineral protective layer on fingernails or hair (for example US 2002/0086039 A1, WO 03/075869 A1), as a preservative (for example WO 01/03650 A2), and as an antiperspirant active ingredient in formulations for sweat reduction (DE 10303553 A1). DE 102007033091 A1 proposes agents for altering the color and/or shape of keratin fibers which comprise bioactive glass in order to reduce the irritation of the scalp that occurs as a result of the strongly alkaline conditions in the course of the color-altering process. The agents for altering the color comprise customary alkalizing agents such as alkali metal or alkaline earth metal hydroxides, ammonia or organic amines. EP 1709997 A1 describes hair straighteners comprising alkali metal or alkali earth metal hydroxides that comprise bioactive glass in order to reduce skin irritation during treatment.

Overall, the previous applications of bioactive glass made use primarily of the property that soluble ions are released from the bioactive glass, wherein the released ions induce a desired effect and/or the surface of the bioactive glass is modified so as to allow bonding to, for example, bones, tissue, tooth dentine or keratin-containing elements such as fingernails.

In the context of the invention it was found that particles of a bioactive glass can surprisingly also be used selectively as alkalizing agent in a colorant, in particular hair colorant. This exploits the effect that monovalent and/or divalent ions from the glass are exchanged for protons (hydrogen ions, H⁺) from the liquid (usually aqueous medium), as a result of which the pH increases overall or only locally.

In the context of the invention, a preferred bioactive glass is one that comprises monovalent cations from the group of alkali metals and/or divalent cations from the group of alkaline earth metals, preferably sodium ions and/or calcium ions, and network-forming components. On coming into contact with a liquid medium, the cations leach from the glass matrix of network-forming components in exchange for protons from the liquid medium, resulting in the establishment of a pH favorable for the absorption of color/change in color on the object to be dyed, in particular hair and/or the skin.

According to an advantageous embodiment, the rate of solubilization, and thus the alkalizing effect, can be adjusted by varying the composition of the bioactive glass.

The rate of reaction of a bioactive glass can be influenced by a number of factors, such as composition (for example SiO₂ content), internal structure of the glass (glass ceramic) and/or porosity of the glass, particle size of the powder particles, etc.

In an advantageous embodiment, the bioactive glass includes SiO₂ as network-forming agent and CaO, and preferably in addition Na₂O and/or P₂O₅.

The SiO₂ content in the glass can be 35% to 75% by weight. The glass can advantageously contain at least 35% by weight, preferably at least 40% by weight, more preferably at least 43% by weight, of SiO₂. An advantageous SiO₂ upper limit can be not more than 75% by weight, not more than 70% by weight, not more than 60% by weight, advantageously not more than 55% by weight, preferably not more than 50% by weight or not more than 48% by weight.

The Na₂O content in the glass can be between 0% and 40% by weight. In advantageous variants, the Na₂O content in the glass can be between 10% and 40% by weight. The glass can advantageously contain at least 10% by weight, preferably at least 15% by weight, preferably at least 18% by weight, more preferably at least 20% by weight, of Na₂O. An advantageous Na₂O upper limit can be not more than 40% by weight, advantageously not more than 35% by weight, preferably not more than 30% by weight or not more than 28% by weight or not more than 26% by weight. There are other advantageous variants with a low Na₂O content in the range of 0% to <10% by weight. Na₂O-free variants are possible and can be advantageous when other ion-releasing alkali metal oxides and/or alkaline earth metal oxides are present instead.

The CaO content in the glass can advantageously be between 10% and 40% by weight. The glass can advantageously contain at least 10% by weight, advantageously at least 15% by weight, preferably at least 18% by weight, more preferably at least 20% by weight, of CaO. An advantageous CaO upper limit can be not more than 40% by weight, advantageously not more than 35% by weight, preferably not more than 30% by weight or not more than 28% by weight or not more than 26% by weight.

The glass can contain P₂O₅ in a proportion of 0% to 30% by weight. The glass can advantageously contain at least 1% by weight, preferably at least 3% by weight, preferably at least 4% by weight, more preferably at least 5% by weight, of P₂O₅. An advantageous P₂O₅ upper limit can advantageously be not more than 30% by weight, advantageously not more than 25% by weight, advantageously not more than 20% by weight, not more than 15% by weight, advantageously not more than 10% by weight, advantageously not more than 9% by weight, preferably not more than 8% by weight or not more than 7% by weight. P₂O₅-free variants are possible and advantageous.

The bioactive glass may advantageously optionally comprise at least one further component preferably selected from the group consisting of Al₂O₃, B₂O₃, MgO, Li₂O, K₂O, Ag₂O, AgI, NaI, TiO₂, ZnO and CaF₂, wherein the sum total of the contents of this further component is advantageously less than 20% by weight, advantageously not more than 18% by weight, advantageously not more than 15% by weight, advantageously not more than 10% by weight, preferably not more than 8% by weight, preferably not more than 5% by weight, preferably not more than 3% by weight.

The bioactive glass used in the context of the invention is toxicologically harmless. Exposure to heavy metals is preferably less than 20 ppm for Pb, less than 5 ppm for Cd, less than 5 ppm for As, less than 10 ppm for Sb, less than 1 ppm for Hg, and less than 10 ppm for Ni.

In an advantageous embodiment, the bioactive glass (in % by weight based on oxide) comprises:

| | |
|---|---|
| SiO₂ | 35 to 75 |
| Na₂O | 0 to 40 |
| CaO | 10 to 40 |
| P₂O₅ | 0 to 30 |

In an advantageous embodiment, the bioactive glass (in % by weight based on oxide) comprises:

| | |
|---|---|
| SiO₂ | 35 to 60 |
| Na₂O | 15 to 40 |
| CaO | 15 to 40 |
| P₂O₅ | 0 to 15 |

In a particularly advantageous embodiment, the bioactive glass (in % by weight based on oxide) comprises:

| | |
|---|---|
| SiO₂ | 40 to 50 |
| Na₂O | 20 to 30 |
| CaO | 20 to 30 |
| P₂O₅ | 2 to 8 |

According to an advantageous embodiment of the present invention, the bioactive glass according to the invention consists of at least 90% by weight, more preferably at least 95% by weight, most preferably at least 99% by weight, of the aforementioned components and preferably of the components SiO₂, Na₂O, CaO, and P₂O₅. In an advantageous variant, the bioactive glass according to the invention consists of at least 90% by weight, more preferably at least 95% by weight, most preferably at least 99% by weight, of the components SiO₂, Na₂O, CaO, P₂O₅, K₂O, and MgO.

The bioactive glass on which the glass powder according to the invention is based may be a known bioactive glass. An example of a particularly advantageous bioactive glass that can be advantageously used in the context of the various aspects of the invention is available under the name Vitryxx® MD01 from SCHOTT AG.

In an advantageous development, the bioactive glass is a ceramized bioactive glass, i.e. the bioactive glass is present as bioactive glass ceramic, i.e. the particles according to the invention may be present in the form of a glass ceramic. Glass ceramic is understood to mean a material that has an amorphous phase and a crystalline phase. Through selective formation of particular crystal phases and the proportions of the crystalline phases and the amorphous phases, it is possible to control the reactivity of the bioactive glass ceramic, i.e. the rate of ion exchange with a liquid medium and thus the establishment of the pH. The higher the reactivity, the less powder is required to establish the desired pH. Experiments have shown that powders or particles of a particular advantageous bioactive glass ceramic show a higher reactivity, i.e. more rapid ion exchange, in an aqueous medium than particles of a bioactive amorphous glass of the same composition and same particle size. However, it can also be the other way around. The change in reactivity is because the different phases formed can have different solubilities in a corresponding medium.

In particular advantageous bioactive glasses, in particular ones that include SiO₂, CaO and Na₂O and optionally P₂O₅, sodium-calcium-silicate crystal phases can be produced in a selective manner.

For control of the reactivity, it may additionally be advantageous when the powder particles are surface-crystallized particles, given that ion exchange takes place primarily at the particle surface. Surface-crystallized, as opposed to bulk-crystallized, means that crystallization starts at the surface of a particle and progresses from there into the interior (into the bulk).

In an advantageous variant, the transformation of the bioactive glass into a bioactive glass ceramic is due to a selective thermal treatment of the powder particles in which the powder is exposed to temperatures in the range between 500° C. to 1000° C. for a period of, for example, 1 to 10 hours, advantageously 1 to 6 hours.

The bioactive glass on which the invention is based may be produced in a known manner in various ways: In an advantageous embodiment, the bioactive glass is a melted glass obtained by melting raw materials. Melted glasses usually have low porosity. In another advantageous variant, the underlying bioactive glass is a sol-gel glass obtained from metal-organic precursor materials via a known sol-gel process. Bioactive sol-gel glasses have relatively high porosity. In another advantageous variant, the underlying bioactive glass may be produced by flame synthesis or microwave synthesis.

In addition to the ingredients described above, different colorants also contain other active ingredients, auxiliaries, and additives well known to persons skilled in the art, the selection of which depends on the respective colorant type. These include for example customary constituents such as water, fats, oils, polymers, thickeners, consistency regulators, perfume oils, solubilizers, complexing agents, surfactants, emulsifiers, etc.

According to a second aspect, the invention relates to the use of particles of bioactive glass in a cosmetic formulation as alkalizing agent for effecting opening of the cuticle layer of hairs and/or swelling of hairs and/or skin. The cosmetic formulation is advantageously a colorant. Preferably, the colorant is used for coloring keratin fibers.

In an advantageous embodiment, the colorant comprises at least one coloring component and/or at least one oxidant.

When used in an oxidative (hair) colorant, the particles of bioactive glass as alkalizing agent can also have the function of catalyzing or initiating coupling reactions of/with color precursors.

In an advantageous embodiment, the cosmetic formulation is a colorant, in particular hair colorant for human hair and/or skin colorant for human skin, as described in detail above in relation to the prior art and the first aspect of the invention. Since the statements made therein on the features and advantageous developments of the invention also apply mutatis mutandis to the use according to the invention, reference is made thereto in order to avoid repetition. This also applies to the description of the mode of action, composition, internal structure, etc. of the bioactive glass or glass ceramic on which the invention is based.

In another advantageous variant, the cosmetic formulation is a shaving composition, for example a shaving foam or a shaving soap, or a depilatory composition, for example a hair removal cream. Such advantageous formulations likewise contain particles of bioactive glass as alkalizing agent that serves to cause hairs and/or skin to swell. For these cosmetic formulations too, the description of the inventive and advantageous features of the particles and effect thereof applies mutatis mutandis to the composition and internal structure of the bioactive glass.

Because particles of bioactive glass have an affinity for keratin fibers, they can advantageously be used in cosmetic and non-cosmetic formulations in which an alkalizing agent is needed to cause swelling of substrates comprising keratin fibers.

A cosmetic formulation, in particular a colorant, may advantageously be a paste, cream, ointment, foam, gel, lotion, suspension, soap, etc. In principle, any formulation capable of accommodating particles of bioactive glass can be suitable. In other words, an advantageous use includes where the cosmetic formulation is or includes a paste, cream, ointment, foam, gel, lotion, suspension, soap, etc.

In an advantageous cosmetic formulation, in particular the colorant, bioactive glass is employed in particle form. The median diameter of the particles (d₅₀ value) is advantageously≤20 μm, advantageously≤15 μm, preferably ≤10 μm, preferably ≤5 μm. The smaller the particle size, the greater the reactive surface area, the greater the degree of ion exchange, and the more strongly the pH can be shifted to alkaline. A lower limit for the d₅₀ value of advantageously 0.1 μm, advantageously 0.25 μm, which can be combined with any of the above upper limits, should not be gone below.

The d₉₉ value of the particle size is in an advantageous variant≤60 μm, advantageously 50 μm, advantageously≤40 μm, advantageously≤30 μm, advantageously≤25 μm, advantageously≤20 μm, preferably ≤15 μm. A lower limit for the d₉₉ value, which can advantageously be combined with any of the above upper limits, can be 1 μm.

In an advantageous embodiment, the proportion of particles of bioactive glass in the cosmetic formulation is 0.1% to 20% by weight, advantageously 0.5% to 15% by weight, advantageously 0.7% to 10% by weight.

The proportion of particles of bioactive glass in the cosmetic formulation, in particular in the colorant, can advantageously be at least 0.1% by weight in order to obtain the desired alkalizing effect. An advantageous lower limit can be at least 0.3% by weight, at least 0.5% by weight, at least 0.7% by weight, at least 0.9% by weight or at least 1% by weight. Some variants may also comprise at least 1.5% by weight or at least 2% by weight of bioactive glass powder. An upper limit of not more than 20% by weight should advantageously not be exceeded, since otherwise the alkalizing effect is too strong, which can result in skin irritation as well as hair and skin damage. An advantageous upper limit can be not more than 15% by weight, not more than 12% by weight, not more than 10% by weight, not more than 8% by weight, not more than 6% by weight or not more than 5% by weight.

In an advantageous embodiment, an alkaline pH in the 8 to 11 range, preferably 9 to 11 or 8 to 10, is established in the cosmetic formulation, in particular the colorant, by particles of bioactive glass as alkalizing agent. In an alternative advantageous variant, the cosmetic formulation, in particular the colorant, has a neutral pH due to particles of bioactive glass as alkalizing agent that is preferably in the 6 to <8 range, preferably in the 6.5 to 7.5 range, and the alkalizing agent is a local alkalizing agent.

The inventors have for the first time recognized that particles of bioactive glass can be used in a colorant as a substitute for known alkalizing agents such as ammonia and/or ammonia alternatives. It may be advantageous when the alkalizing agent used contains as small a proportion as possible of other alkalizing agents in addition to particles of bioactive glass. This allows the disadvantages of ammonia and known ammonia alternatives to be avoided. According to a preferred embodiment of the use, a total of less than 5% by weight, preferably less than 3% by weight, of other alkalizing agents (ammonia and ammonia alternatives) is used in the cosmetic formulation. Preference is given to using less than 2% by weight, preferably less than 1% by weight, of other alkalizing agents (ammonia and ammonia alternatives). It is particularly advantageous when particles of bioactive glass are used as sole alkalizing agent, i.e. there is complete replacement of ammonia and ammonia alternatives. In such an advantageous use, the cosmetic formulation, in particular the colorant, is free of ammonia and/or ammonia alternatives as alkalizing agent (for the definition of “free of”, see above).

In an advantageous use, the cosmetic formulation, in particular the colorant, in use has a pH in the 6 to <8 range, preferably in the 6.5 to 7.5 range. As described above, an advantageous colorant comprising particles of bioactive glass may—depending on its use concentration—in use have a neutral pH, this being understood to mean in the context of the invention a pH range of 6 to <8, preferably 6.5 to 7.5, whereas known alkalizing agents determine the pH of the colorant overall, wherein a pH of 8 to 11 is usually established. This is possible because bioactive glass is reactive and special particles of bioactive glass in contact with the hair and/or skin locally make the pH more basic and in doing so effect opening of the cuticle layer of the hairs and cause hair and/or skin to swell and/or enable reactions resulting in color formation, while the pH of the colorant is neutral overall. In such variants, the particles of bioactive glass act as a local alkalizing agent.

In an advantageous use, the bioactive glass includes SiO₂ as network-forming agent and CaO and preferably comprises Na₂O and/or P₂O₅ (see detailed description of the composition in relation to the first aspect of the invention).

In an advantageous embodiment, the particles include a bioactive glass ceramic. In other words, the cosmetic formulation, preferably the colorant, in an advantageous use comprises particles of ceramized bioactive glass.

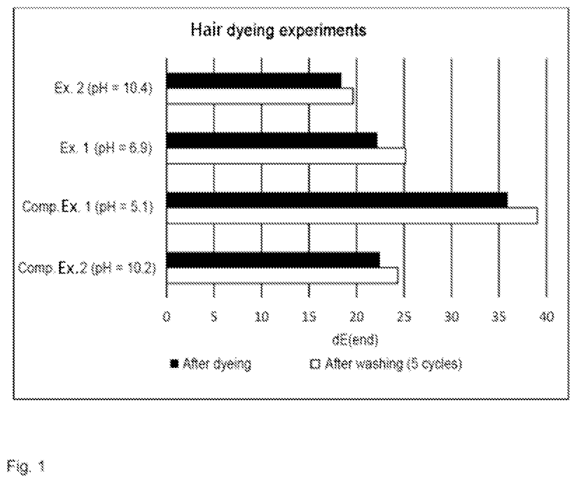

The invention will be described in more detail hereinbelow with reference to FIG. 1 and the embodiment examples by way of example and without limitation thereto.

The FIGURE shows:

FIG. 1: Results of colorimetric measurements in coloring experiments

### EXAMPLES

The invention is described by way of example hereinbelow with reference to a permanent hair colorant, without being limited thereto.

Table 1 below shows compositions (data in units of grams, g) for two embodiment examples (Ex.) and two comparative examples (Comp. Ex.) of a “coloring component” of a two-component colorant.

TABLE 1
Composition of coloring components [in g]

| | Comp. | Comp. | | |
|---|---|---|---|---|
| | Ex. 1 | Ex. 2 | Ex. 1 | Ex. 2 |
| Water phase | | | | |
| Water | 14.70 | 14.70 | 14.70 | 14.70 |
| Xanthan gum | 0.07 | 0.07 | 0.07 | 0.07 |
| Propylene glycol | 0.60 | 0.60 | 0.60 | 0.60 |
| Sodium sulfite | 0.09 | 0.09 | 0.09 | 0.09 |
| Ascorbic acid | 0.02 | 0.02 | 0.02 | 0.02 |
| Sodium laureth sulfate | 0.11 | 0.11 | 0.11 | 0.11 |
| Coloring components | | | | |
| 2,5-Diaminotoluene sulfate | 0.20 | 0.20 | 0.20 | 0.20 |
| Resorcinol | 0.15 | 0.15 | 0.15 | 0.15 |
| p-Aminophenol | 0.05 | 0.05 | 0.05 | 0.05 |
| 4-Amino-2-hydroxytoluene | 0.04 | 0.04 | 0.04 | 0.04 |
| m-Aminophenol | 0.03 | 0.03 | 0.03 | 0.03 |
| Fatty phase | | | | |
| Cetyl stearyl alcohol | 1.20 | 1.20 | 1.20 | 1.20 |
| 2-Octyldodecan-1-ol | 0.80 | 0.80 | 0.80 | 0.80 |
| Stearic acid | 0.32 | 0.32 | 0.32 | 0.32 |
| Palmitic acid | 0.32 | 0.32 | 0.32 | 0.32 |
| Alkalizing agent | | | | |
| 2-Ethanolamine | — | 0.90 | — | — |
| Ammonium hydroxide | — | 0.40 | — | — |
| Bioactive glass (BAG) | — | — | 1.3 H₂O + | 1.3 H₂O + |
| | | | 0.2 g BAG | 1 g BAG |
| Perfume oil (optional) | 0.05 | 0.05 | 0.05 | 0.05 |

The compositions shown above differ only with regard to the alkalizing agent.

Comp. Ex. 1 does not contain an alkalizing agent in the coloring component. Comp. Ex. 2 comprises a mixture of 2-ethanolamine and ammonium hydroxide as alkalizing agent, whereas in the embodiment examples (Ex. 1 and 2) only particles of bioactive glass are employed as alkalizing agent. The d₅₀ value of the particles used is 4 μm in both embodiment examples and the d₉₉ value is <15 μm. In both embodiment examples, the same bioactive glass is used, which includes SiO₂ as network-forming agent and CaO and here also comprises Na₂O and P₂O₅. The glass Vitryxx® MD01, which is commercially available, was used by way of example for the embodiment examples shown here. The invention is of course not limited to this specific glass. Other bioactive glasses and glass ceramics and/or other particle sizes may lead to comparable results.

Based on the coloring component of the two-component colorant, the proportion of particles of bioactive glass in the first embodiment example (Ex. 1) is 1% by weight and in the second embodiment example (Ex. 2) 5% by weight.

The coloring component was produced as follows:
1) Melting of the fat phase at 70° C.
2) Homogenizing the thickener (here xanthan gum) and water at 70° C.
3) Adding and dissolving the water-soluble constituents and the dyes
4) Adding the alkalizing agent (omitted in Comp. Ex. 1)
5) Adding the fat phase to the water phase
6) Homogenizing with cooling (up to 50° C.)
7) Adding a fragrance
8) Cooling to room temperature

Between the various steps, the mixture was always homogenized by stirring.

After the production of the four coloring components, the pH was in each case measured over a period of three days. This found that the pH in a coloring component swiftly becomes established at a stable value. With regard to Ex. 1 and 2, this means that the ion exchange reactions between the particles of bioactive glass and the aqueous medium swiftly reach a state of equilibrium after which the pH essentially no longer changes.

The hair coloring experiments were performed on samples of blond human hair. For this purpose, ready-to-use colorants were prepared with the coloring components listed in Table 1 (“component 1” of the two-component colorant) by mixing with “component 2” (oxidation component) of the two-component colorant. A 3% by weight aqueous H₂O₂ solution was in each case used as “component 2”. For each 1 g of coloring component, 1 g of “component 2” was used. Each colorant was tested on two separate hair samples.

The colorant was applied to the hair sample as evenly as possible and the exposure time in the hair was 30 minutes. The colorant was then washed out with tap water for 3 minutes, the hair sample was dried with a hairdryer, and the coloring performance was measured by colorimetry. The dyed hair sample was then washed several times. In each washing cycle, the hair sample was washed with a standard commercial shampoo for 2 minutes and rinsed with tap water for seconds, dried with a hairdryer, and measured again with a colorimeter. A total of 5 washing cycles was carried out. This was the procedure for all hair samples.

To quantify the coloring performance directly after coloring and the color retention after washing, the hair samples were measured colorimetrically. Colorimetric methods are generally well known. In the present case, a “Lorentzen & Wettre Datacolor Elrepho spectrophotometer” was used, which conforms to the ISO2469 standard and was used with d/0° geometry. The light source was a pulsed xenon lamp with an “almost D65” filter.

The measurement principle is based on the CIELAB color space. This color model is standardized in EN ISO 11664-4 (part 4). Three axes (green-red; yellow-blue; black-white) define a three-dimensional space. Every color can be defined by the coordinates a* (red-green), b* (yellow-blue) and L* (brightness). This color system is particularly suitable for expressing color differences. The distance from the origin is referred to as dE, also termed “ΔE” or “delta E”, and is calculated according to equation (1):

$$dE = \sqrt{{a}^{2} + {b}^{2} + {L}^{2}}$$

where “a”, “b”, and “L” are the coordinates of the color space.

With regard to the dE values, the smaller the dE, the higher the color intensity. Here it is assumed that, for the hair color produced, the black-white proportion, i.e. “L” in the above formula, is the most important factor in the calculation of dE. Therefore, a low value for “L” and consequently a small dE indicates darker hair, i.e. more intense coloration. This applies in particular to the observation period specified below.

Before the start of the sample measurement, the instrument was calibrated using instrument-specific standards (Black Cavity, White Calibration, UV Calibration).

A dried hair sample was placed on the sample holder. For each measurement run, the hair sample was measured in 5 different positions. The computer calculated from the 5 individual measurements average values for the color coordinates a*, b*, and L*. These values were used to calculate the associated dE (Equation 1). For each hair sample, two such measurement runs were carried out and the average value for dE was calculated from the two results. Each colorant was tested on a total of two hair samples and the two dE values determined therefrom were in each case used to determine an average value as the end result dE_{(end)}.

The measurements were performed and evaluated at a constant temperature and by the same person.

The colorimetric results are summarized in Table 2 below. “dE_{(end)} directly” refers to the end result for dE directly after coloring and “dE_{(end)} 5 washes” refers to the end result for dE after 5 washing cycles. The measured pH in the coloring component is also stated.

TABLE 2
Dyeing results

| | dE_{(end)} directly | dE_{(end)} 5 washes | pH |
|---|---|---|---|
| Comp. Ex. 1 | 35.9 | 39.0 | 5.1 |
| Comp. Ex. 2 | 22.5 | 24.3 | 10.2 |
| Ex. 1 | 22.4 | 25.1 | 6.9 |
| Ex. 2 | 18.4 | 19.6 | 10.4 |

In general, it can be seen from Table 2 that, for the comparison examples (Comp. Ex.) and examples (Ex.), the determined “dE_{(end)}” value increased after washes compared to the baseline value “dE_{(end)} directly”, i.e. the color intensity decreased in all cases. However, the smaller the difference between the values “dE_{(end)} directly” and “dE_{(end)} 5 washes”, the higher the color retention after washing.

Comp. Ex. 1 did not contain any alkalizing agent in the coloring component; the pH was slightly acidic. The color intensity after coloring was much lower (dE_{(end)}=35.9) compared to Comp. Ex. 2, Ex. 1, and Ex. 2. The color durability after 5 washing cycles was also considerably poorer (dE_{(end)}=39.0).

When coloring hair with a colorant based on Comp. Ex. 2, which comprised ammonia and 2-ethanolamine and had a pH of 10.2, the color intensity directly after coloring had a value of dE_{(end)}=22.5, which had changed after 5 washing cycles to dE_{(end)}=24.3, denoting a reduction in color intensity.

Ex. 1 employed 1% by weight of particles of bioactive glass as alkalizing agent. With regard to the color intensity directly after coloring and after 5 washing cycles, the coloring result and durability obtained corresponded to those in Comp. Ex. 2: a change from dE_{(end)}=22.4 directly after coloring to dE_{(end)}=25.1 after 5 washing cycles.

The colorimetric measured values show that particles of bioactive glass as alkalizing agent according to the invention, even when present in a relatively low proportion in a colorant, achieve a comparable result in respect of coloring performance and color durability to that obtained for known colorants comprising ammonia or ammonia alternatives. A particular point to note is that, while coloring performance and durability are comparable, the pH of the advantageous colorant is—in contrast to known colorants—neutral (pH=6.9) during coloring (see Table 2, Ex. 1). A content of 1% by weight of particles of bioactive glass is thus an example of a local alkalizing agent that locally has an alkalizing effect in hair comparable to that of ammonia or ammonia alternatives, the colorant having a neutral pH overall. The neutral pH ensures that the skin is less irritated.

When the proportion of particles of bioactive glass in the colorant is increased—for example to 5% by weight based on the coloring component; see Ex. 2—the coloring performance and color durability are even improved compared to known colorants comprising ammonia or ammonia alternatives: In Ex. 2, the dE_{(end)} values directly after coloring (dE_{(end)}=18.4) and after 5 washing cycles (dE_{(end)}=19.6) are in each case lower than for Comp. Ex. 2, whereas the pH during coloring is comparable (pH 10.4). This demonstrates that an advantageous colorant comprising particles of bioactive glass as alkalizing agent achieves at the same colorant pH a better coloring result and better color durability than known colorants.

The results of the colorimetric investigations of the dyed hair samples are shown in graph form in FIG. 1.

1. A colorant that comprises an alkalizing agent for effecting opening of the cuticle layer of hairs and/or swelling of hairs and/or skin, the alkalizing agent comprising particles of bioactive glass, wherein the colorant has a DH in the 6 to <8 range and comprises particles of bioactive glass as a local alkalizing agent.

2. The colorant as claimed in claim 1, wherein a total amount of ammonia and ammonia alternatives in the colorant is less than 3% by weight.

3. The colorant as claimed in claim 1, wherein the colorant is free of ammonia and/or free of ammonia alternatives.

4. The colorant as claimed in claim 1, wherein the colorant comprises at least one coloring component and/or at least one oxidant.

5-6. (canceled)

7. The colorant as claimed in claim 1, wherein a d₅₀ value of a size of the particles of bioactive glass is ≤20 μm and/or wherein a d99 value of a size of the particles of bioactive glass is ≤60 μm.

8. (canceled)

9. The colorant as claimed in claim 1, wherein a proportion of particles of bioactive glass in the colorant is 0.1% to 80% by weight.

10. (canceled)

11. The colorant as claimed in claim 1, wherein the bioactive glass includes SiO₂ as network-forming agent and CaO.

12. A cosmetic formulation comprising particles of bioactive glass, as a partial or complete substitute for ammonia and/or ammonia alternatives, as alkalizing agent for effecting opening of the cuticle layer of hairs and/or swelling of hairs and/or skin.

13. The cosmetic formulation of claim 12, wherein the cosmetic formulation is a colorant that comprises at least one coloring component and/or at least one oxidant, wherein the cosmetic formulation has a pH in the 6 to <8 range.

14. The cosmetic formulation of claim 12, wherein a total of less than 3% by weight of ammonia and ammonia alternatives is used in the cosmetic formulation as alkalizing agents.

15. The cosmetic formulation of claim 12, wherein particles of bioactive glass are used as sole alkalizing agent.

16-17. (canceled)

18. The cosmetic formulation of claim 12, wherein a d₅₀ value of a size of the particles of bioactive glass is ≤20 μm and/or wherein a d99 value of a size of the particles of bioactive glass is 60 μm.

19. (canceled)

20. The cosmetic formulation of claim 12, wherein a proportion of particles of bioactive glass in the cosmetic formulation is 0.1% to 8% by weight.

21. (canceled)

22. The cosmetic formulation of claim 12, wherein the bioactive glass includes SiO₂ as network-forming agent and CaO.

23. The cosmetic formulation of claim 12, wherein the cosmetic formulation is a paste, a cream, an ointment, a foam, a gel, a lotion, suspension, a soap, or the like.

24. The colorant as claimed in claim 11, wherein the bioactive glass further comprises Na₂O and/or P₂O₅.

25. The cosmetic formulation of claim 12, wherein the cosmetic formulation is a shaving composition or a depilatory composition and wherein the cosmetic formulation has a pH in the 6 to <8 range or wherein the cosmetic formulation has a pH in the 8 to 11 range.

26. The cosmetic formulation of claim 22, wherein the bioactive glass further comprises Na₂O and/or P₂O₅.
